(19) United States
(12) Patent Application Publication
(10) Pub. No.: US 2010/0162779 A1
(43) Pub. Date: Jul. 1, 2010

(54) Apparatus And Method For Composting Organic Matter

(76) Inventor: Itzhak Aviram, Moshav Shdema (IL)

Correspondence Address: Professional Patent Solutions, P.O. BOX 654, HERZELIYA PITUACH, 46105, IL

(21) Appl. No.: 12/647,427

(22) Filed: Dec. 25, 2009

Related U.S. Application Data
(60) Provisional application No. 61/140,859, filed on Dec. 25, 2008.

(51) Int. Cl. C05F 11/08; C12M 1/10; C12M 1/04

(52) U.S. Cl. 71/9; 435/290.1; 435/290.3

(57) ABSTRACT

Disclosed are apparatus and methods for composting organic matter. According to some embodiments, the apparatus may include a reactor adapted to receive organic matter in a reaction chamber of the reactor through a first sealable opening in the reactor. At least one protruding structure protruding inward from an inner surface of said reaction chamber may include a cavity allowing flow of gases into and out of the reaction chamber.

### FIELD OF THE INVENTION

The present invention generally relates to the fields of biodegrading, biodegrading of organic matter, waste, and compost. More specifically, the present invention relates to an apparatus and method for composting such matter.

### BACKGROUND

Compost is a combination of decomposed/biodegraded organic matter, such as plant and animal materials and other organic materials that have decomposed largely through aerobic decomposition into a rich black soil.

Compost soil is very rich soil and may be used for different purposes, such as in gardening, landscaping, horticulture, agriculture, etc. Compost soil itself is beneficial for the land in many ways, including as a soil conditioner, a fertilizer to add vital humus or humic acids, and as a natural pesticide for soil. In addition, compost soil may be useful for erosion control, land and stream reclamation, wetland construction, and as landfill cover.

Composting is the purposeful biodegradation of organic matter, such as yard and food waste. The biodegradation/decomposition is performed by micro-organisms, mostly bacteria, but also yeasts and fungi. In low temperature phases a number of macro-organisms, such as springtails, ants, nematodes, isopods and red wigglers also contribute to the process, as well as soldier fly, fruit flies and fungus gnats. There are a wide range of organisms in the decomposer community.

Compostable matter is capable of being completely broken down under the action of microorganisms into carbon dioxide, water and biomass. It may take a very long time for some material to biodegrade depending on its environment (e.g. wood in an arid area versus paper in water), but it ultimately breaks down completely.

Compostable matter biodegrades substantially under specific composting conditions. It is metabolized by the microorganisms, being incorporated into the organisms or converted into humus. The size of the material is a factor in determining compostability, and mechanical particle size reduction can speed the process.

Composting is an ancient human practice, mention of which can be found in the Bible several times and can be traced to Marcus Cato, a farmer and scientist who lived in Rome 2,000 years ago. Cato viewed compost as the fundamental soil enhancer, essential for maintaining fertile and productive agricultural land. He stated that all food and animal wastes should be composted before being added to the soil. By the 19th century in America, most farmers and agricultural writers knew about composting.

Composting upcycles organic kitchen and yard waste and manures into an extremely useful humus-like, soil end product, permitting the return of vital organic matter, nutrients, and particularly bacteria, that are vital to plant nutrition through the soil. Managed aerobic composting arranges environmental conditions so they are optimal for the natural processes to take place.

Long used in subsistence farming and home gardening for creating garden-ready soil, composting is becoming increasingly important and better understood as a tool for reducing municipal solid waste, and reducing the amount of green waste going into landfills.

Land filling organic waste takes up landfill space needed for other wastes. In addition, organic waste which is land filled breaks down very slowly due to the lack of oxygen. As it decomposes, it produces methane gas, an important greenhouse gas, making the reduction of organic waste being land filled a key element in the fight against climate change.

Another standard means for disposal of organic waste is by incineration. However, incinerating moist organic waste is inefficient and results in poor combustion, which disrupts the energy generation of the facility and increases the pollutants that need to be removed by the pollution-control devices. Composting these wastes is a more effective and usually less expensive means of managing organic wastes.

Yard and food wastes make up approximately 30% of the waste stream in the United States. Composting most of these waste streams would reduce the amount of Municipal Solid Waste requiring disposal by almost one fourth, while at the same time provide a nutrient-rich soil amendment. Compost added to gardens improves soil structure, texture, aeration, and water retention. When mixed with compost, clay soils are lightened, and sandy soils retain water better. Mixing compost with soil also contributes to erosion control, soil fertility, proper pH balance, and healthy root development in plants.

Studies have shown that domestic composting can divert an average of 700 lbs. of organic matter per household per year from the waste stream. Municipal composting carries a greater environmental cost, but not nearly as high as if leaf and yard waste are disposed of by conventional means (e.g. land filling and incineration). Composting is an excellent way to avoid wasting useful natural resources, and creating environmental problems, while at the same time producing a high quality and inexpensive soil amendment.

Traditionally, composting was to pile organic materials and let them stand for about a year, or until the next planting season, at which time the materials would be ready for soil application. The main advantage of this method is that little working time or effort is required from the composter and it fits in naturally with agricultural practices in temperate climates. Disadvantages (from the modern perspective) are that space is used for a whole year, some nutrients might be leached due to exposure to rainfall, and disease producing organisms, some weeds, weed seeds and insects may not be adequately controlled.

There are many modern proponents of rapid composting which attempt to correct some of the perceived problems associated with traditional, slow composting. Many such short processes involve a few changes to traditional methods, including smaller, more homogenized pieces in the compost, controlling carbon to nitrogen ratio at 30 to 1 or less, and monitoring the moisture level more carefully. However, none of these parameters differ significantly from early writings of Howard and Balfour, suggesting that in fact modern composting has not made significant advances over traditional organic practices.

There is a popular expression: “compost happens”, but it is helpful to accommodate for optimal as possible circumstances for large amounts of organic waste to decompose quickly and efficiently, with better conservation of useful nutrients and mass. Uncontrolled composting is when compost “happens”, and although that may be functional in some circumstances, as with forest floor detritus, a neglected mass of organic kitchen and yard wastes will more likely result in potentially hazardous and harmful by-products long before it will result useful compost.

### SUMMARY OF THE INVENTION

The present invention is an apparatus and method for composting organic matter. According to some embodiments of the present invention, there is provided an apparatus for composting organic matter including a reactor which may receive organic matter in a reaction chamber of the reactor through a first sealable opening in the reactor. The reactor may also include at least one protruding structure, protruding inward from an inner surface of the reaction chamber. The at least one protruding structure may include a cavity allowing for free flow of gases into and out of the reaction chamber.

According to some embodiments of present invention, the protruding structure may be in the shape of a cylinder or a cone or any other suitable shape. The protruding structures may include a cavity, channel or other pathway for providing a passage for gas between an opening on a surface of the protruding structure and an opening at or near a base of the protruding structure. The base of the protruding structure may connect to a connection point on an inner surface of the reaction chamber, which connection point may include an opening to the outside of the reactor, thereby allowing gas a passageway between a surface on the protruding structure and outside of the reactor. According to some embodiments of the present invention, there may be provided a set of protruding structures, at least some of which having a length different from at least one other protruding structure. Each protruding structure may include one or a set of openings connected to an inner cavity, wherein each opening may be located at a different point on the surface of the protruding structure, such as at different distances from the structure's contact point with the inner surface of the chamber, for example one near the base of the structure and one at the far end of the structure.

According to some embodiments of the present invention, the combination of the cavities provided in some or all of the protruding structures coupled with the openings on the structures and at the connection points of the chamber may facilitate the introduction of reactive gases (e.g. Oxygen) from outside the chamber into the chamber. This combination may also provide for waste gases (e.g. Carbon Dioxide) to be allowed to leave the chamber, thereby providing circulation of gases to and from the reaction chamber. The arrangement of the openings and cavities may be such that although gases are allowed to circulate, substantially all of the material being processed inside the chamber is retained within the reaction chamber.

According to some embodiments of the present invention, the reactor and/or the reaction chamber may be substantially spherical, substantially oval or substantially egg shaped. The reactor, including the reaction chamber, may be adapted to rotate along multiple axes. The protruding structures may be shaped and serve to churn, break-up and/or mix the matter being processed in the chamber when the reactor is rotated. Gas pathways formed by a combination of one or more openings on the protruding structure surface, one or more cavities within the protruding structure itself and an opening at a connection point to the inner surface of the reaction chamber may facilitate the introduction of gases from outside the reactor into the matter being processed. According to further embodiments of the present invention, when the reactor is rotated, reactive gases from outside the reactor are substantially evenly and/or uniformly introduced via the protruding structures into the matter being processed.

According to some embodiments of the present invention, the reactor may also include at least one draining opening in an inner surface of the reaction chamber, thereby allowing drainage of fluids from the reaction chamber.

According to further embodiments of the present invention, the reaction chamber may include a second sealable opening, which second sealable opening may have a different diameter from a diameter of the first sealable opening. According to one embodiment of the present invention, the second sealable opening may be at a location different from the location of the first sealable opening. According to further embodiments of the present invention, the second sealable opening may be within a lid or cover for the first sealable opening. The second sealable opening may be functionally associated with a lid or cover of its own.

According to some embodiments of the present invention, there may be provided a base structure for supporting the reactor, wherein the base structure may include: (1) a foundation adapted to sit on the ground, and (2) a ring shaped, donut shaped or other shaped frame or receptacle adapted to receive and support the reactor. The base may include on or near a contact surface with the reactor at least one wheel, at least one ball barring assembly or any other functionally suitable mechanism for facilitating rotation of the reactor, and thereby its reaction chamber, along one or more axes. According to further embodiments of the present invention, the reactor and its reaction chamber may be rotated substantially freely along multiple axes.

According to yet further embodiments of the present invention, there may be provided a shredding mechanism adapted to at least partially shred organic matter as it is introduced into the reaction chamber. The shredding mechanism may include a sleeve passing through a surface of the reaction chamber; at least one blade, and a rotatable handle positioned outside the sleeve and adapted to rotate the at least one blade. The shredding mechanism may be integral or otherwise associated with the reactor, the reaction chamber and/or an opening of the reactor. According to some embodiments of the present invention, the handle may be detachable from a socket of the shredding mechanism, which socket may be on an outer surface of the reactor. According to further embodiments of the present invention, substantially all of the shredding mechanism may be removable from the reactor, and may be adapted to enter the reaction chamber through a sealable opening of the reactor.

According to some embodiments of the present invention, the reactor may be comprised of interlocking panels which may be locked together to form the outer surface and reaction chamber of the reactor. The protruding structures may be adapted to lock into connection points on an inner surface of one or more of the panels. The shredding mechanism may lock into a connection bracket on an inner or outer surface of one or more of the panels. One or more of the panels may include a drainage opening, such as a sealable drainage opening.

According to further embodiments of the present invention, there may be provided a temperature sensing/reading apparatus adapted to sense and display a temperature of the chamber environment and/or of the matter being processed inside the chamber. According to further embodiments of the present invention, a sensing portion of the temperature reading apparatus may be located at or near the center of the reaction chamber, for example at or near the end of one of the protruding structures. A display portion of the temperature reading apparatus may be placed on an outer surface of the reactor. The display portion may include an indicator line or indicator region associated with low reactivity within the reaction chamber. According to some embodiments of the present invention, low reactivity may indicate that the reactor should be rotated in order to churn the matter being processed and/or to introduce additional reactive gases into the chamber. Alternatively, low reactivity after rotation may indicate that the processing of the matter inside the chamber is substantially complete.

According to further embodiments of the present invention, there may be provided a humidity reading apparatus adapted to sense and display a humidity level of the chamber environment and/or of the matter being processed inside the reactor. According to further embodiments of the present invention, a sensing portion of the humidity reading apparatus may be located at or near the center of the reaction chamber, for example at or near the end of one of the protruding structures. A display portion of the humidity reading apparatus may be placed on an outer surface of the reactor. The display portion may include an indicator line or indicator region associated with humidity related low reactivity within the reaction chamber due to either excessive or insufficient moisture. According to some embodiments of the present invention, humidity related low reactivity may indicate that the reactor should be rotated in order to churn the matter being processed, to introduce additional reactive gases into the chamber, and/or to facilitate drainage of the chamber. Alternatively, humidity related low reactivity may indicate that fluids need to be introduced into the chamber.

It should be understood by one of ordinary skill in the art that any of the functions performed by any of the above mentioned structures may be performed by analogous structures. Furthermore, the functions mentioned above may be the subject of method claims, disassociated from the specific structures.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a perspective view of an exemplary reactor/base assembly according to some embodiments of the present invention including an exemplary reactor sitting on an exemplary reactor base;

FIG. 1B is a perspective view of the exemplary reactor of FIG. 1A;

FIG. 1C is a top view of the exemplary base of FIG. 1A;

FIG. 2A is a perspective view of the inside of a portion of a reactor according to some embodiments of the present invention;

FIG. 2B is a perspective view of the inside of a portion of a reactor according to some embodiments of the present invention including protruding structures;

FIG. 3 is an external perspective view of a portion of a reactor according to some embodiments of the present invention including a first opening, a first lid for the first opening and a second opening on the first lid, which second opening is functionally associated with a second lid;

FIG. 4A is an external perspective view with cutaways of a portion of a reactor according to some embodiments of the present invention including an exemplary shredding mechanism or assembly having blades, one or more shafts, a sleeve, a socket and a handle for connecting to the socket and turning one of the shafts;

FIG. 4B is an internal perspective view of a portion of a reactor according to some embodiments of the present invention including a temperature reading/sensing apparatus having an internal sensor and an external display; and

FIG. 4C is an internal perspective view of a portion of a reactor according to some embodiments of the present invention including a humidity reading/sensing apparatus having an internal sensor and an external display. Also shown in FIG. 4C is a drainage opening and drainage opening plug.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known structures, apparatuses, methods, and components have not been described in detail so as not to obscure the present invention.

The present invention is an apparatus and method for composting organic matter. According to some embodiments of the present invention there is provided an apparatus for composting organic matter including a reactor which may receive organic matter in a reaction chamber of the reactor through a first sealable opening in the reactor. The reactor may also include at least one protruding structure, protruding inward from an inner surface of the reaction chamber. The at least one protruding structure may include a one or more openings and cavity allowing for flow of gases into and out of the reaction chamber.

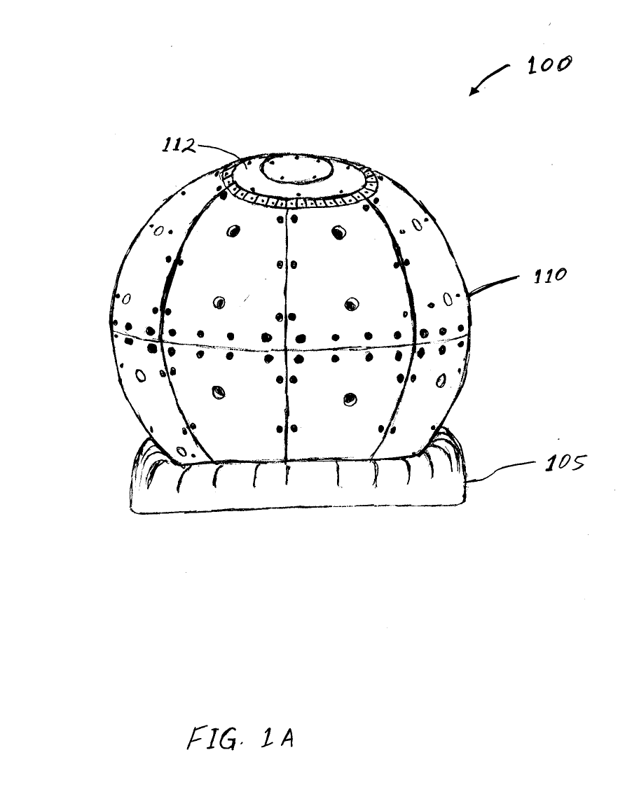

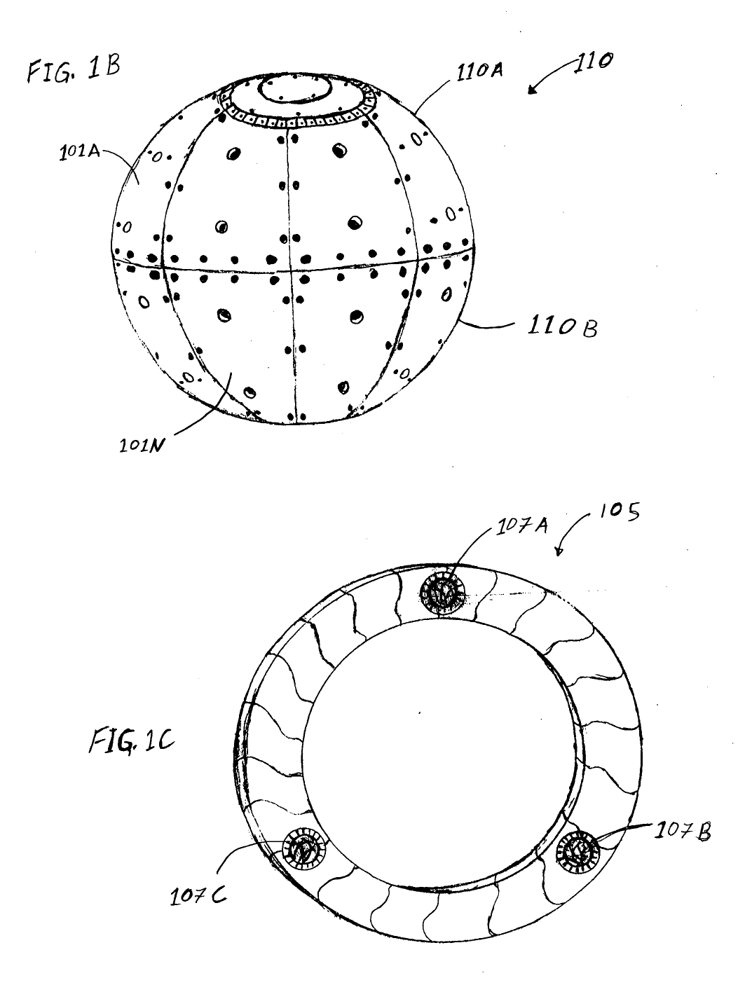

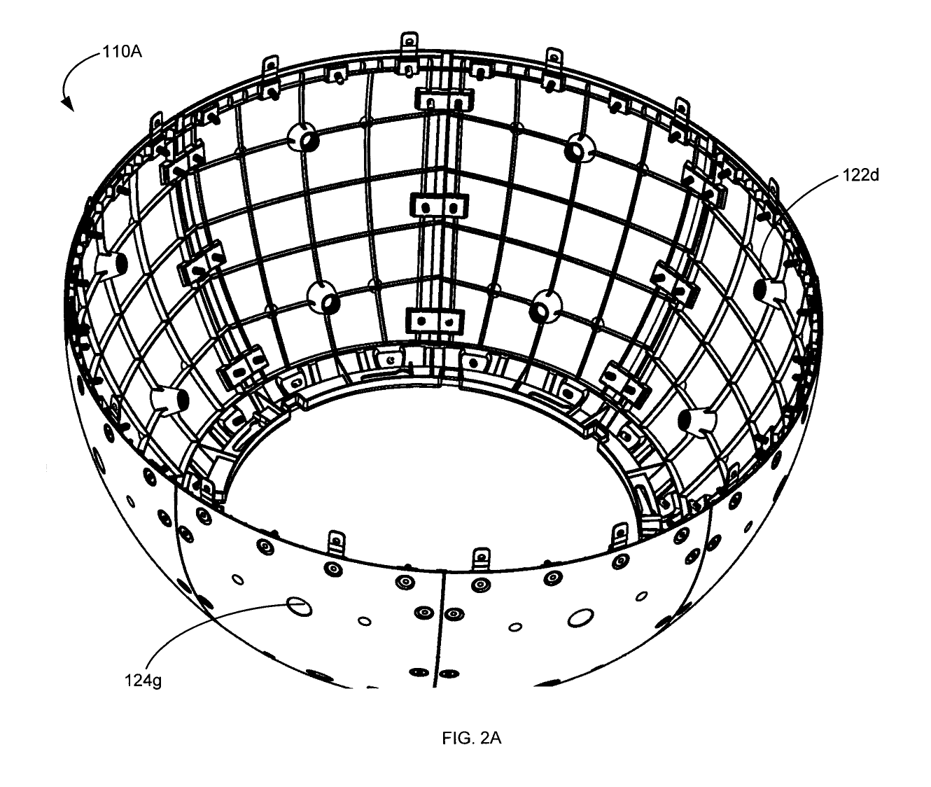

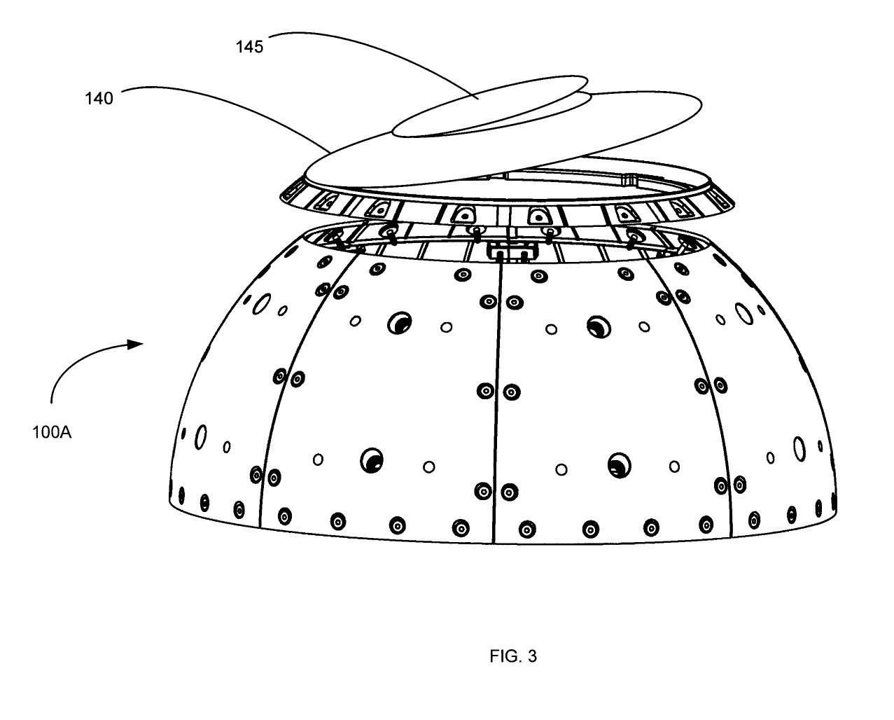

Turning now to FIG. 1A, there is shown a perspective view of an exemplary reactor/base combination 100 including an exemplary reactor 110 sitting on an exemplary reactor base 105 according to some embodiments of the present invention. A top portion of the reactor 110A may include a sealable opening 112. The sealable opening 112 may be sealed with a first lid 140 as shown in FIG. 3. According to embodiments of FIG. 3, the first lid 140 may include a second sealable opening which may be sealable by a second lid 145. Returning to FIG. 1A, the base 105 includes a receptacle portion for receiving the reactor, which receptacle includes a wheel or ball bearing 107 assembly allowing the reactor to be rotated along multiple axes.

Turning now to FIG. 1B, there is shown a perspective view of the exemplary reactor 100 of FIG. 1A. The reactor of FIG. 1A is composed of multiple panels 101A through 101N. The panels interlock and are fastened to each other.

Turning now to FIG. 1C, there is shown a top view of the exemplary base 105 of FIG. 1A. The base 105 includes a wheel or ball bearing assembly 107, including wheels or ball bearings 107A, 107B and 107C.

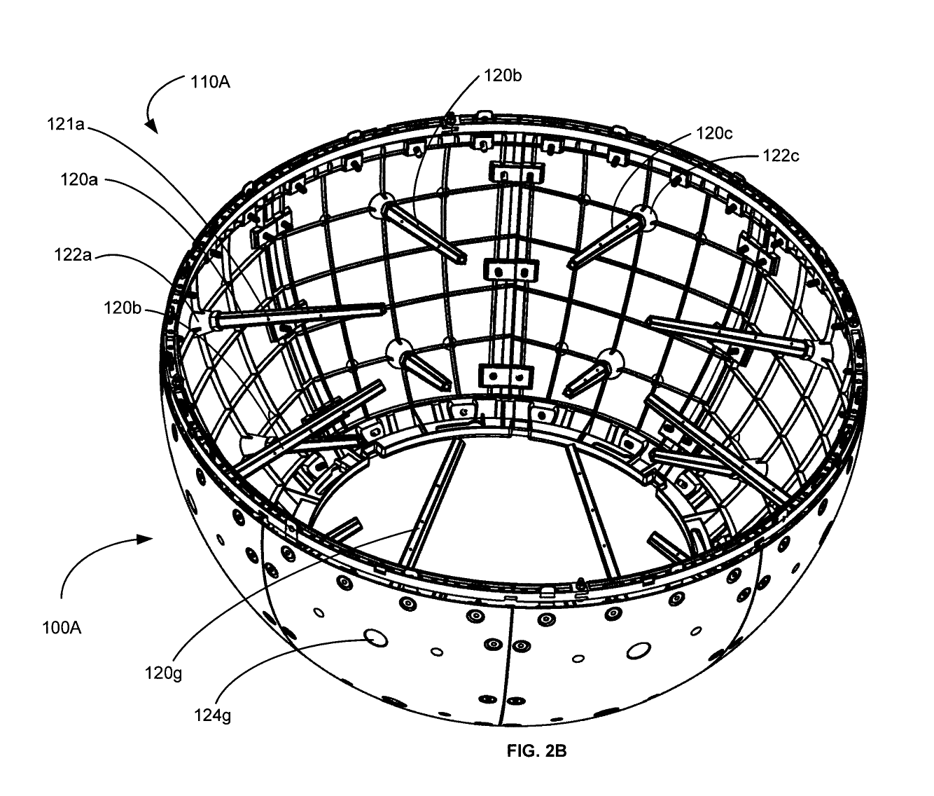

Turning now to FIG. 2A, there is shown a perspective view of the inside of a portion of the reactor 110A according to some embodiments of the present invention. Visible in figures is an inner surface of a chamber of the reactor including protruding structure connection points such as the one designated 122d. Also visible in FIG. 2A is an outer surface including openings such as the one designated 124g. Outer surface openings 124 pass through the reactor and connect with openings in the connection points 122, thereby forming a gas passageway.

Turning now to FIG. 2B, there is shown a perspective view of the inside of a portion of a reactor 110A according to some embodiments of the present invention including protruding structures 120. The protruding structures connect to connection points 122 and openings in the structures 121 form a gas passageway to the outside of the reactor portion 110A through a cavity (not visible) in the structure 120 (e.g. the structures are hollow). The gas passageway from the structure openings 121 extends outside of the reactor portion 110A through: the structure cavity, an opening at the connection point and an opening on the outside surface of the reactor 124.

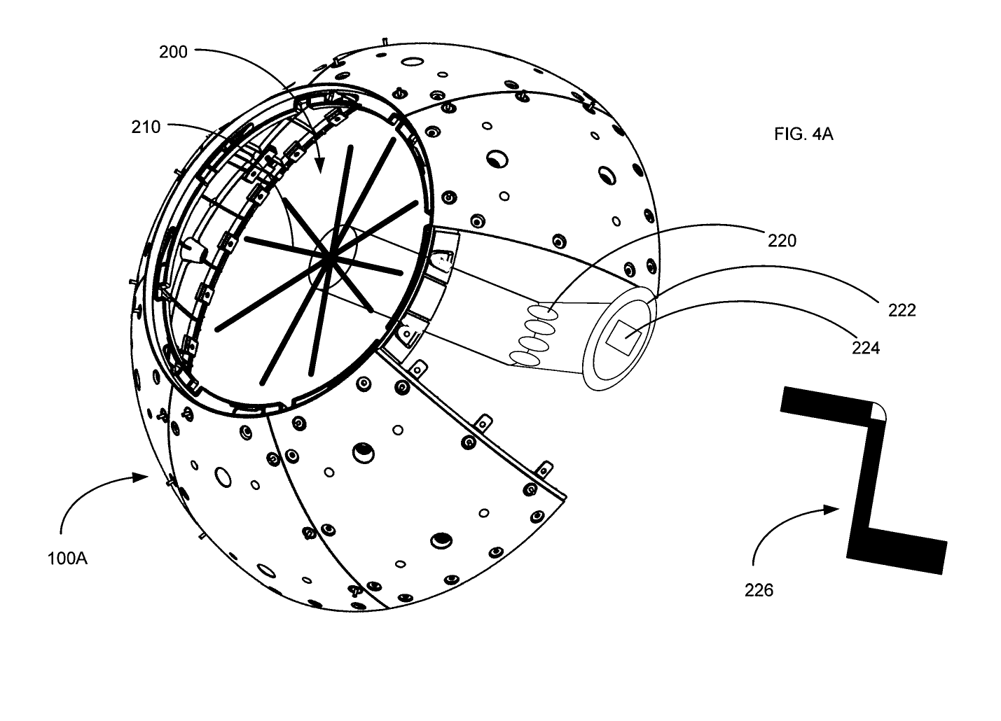

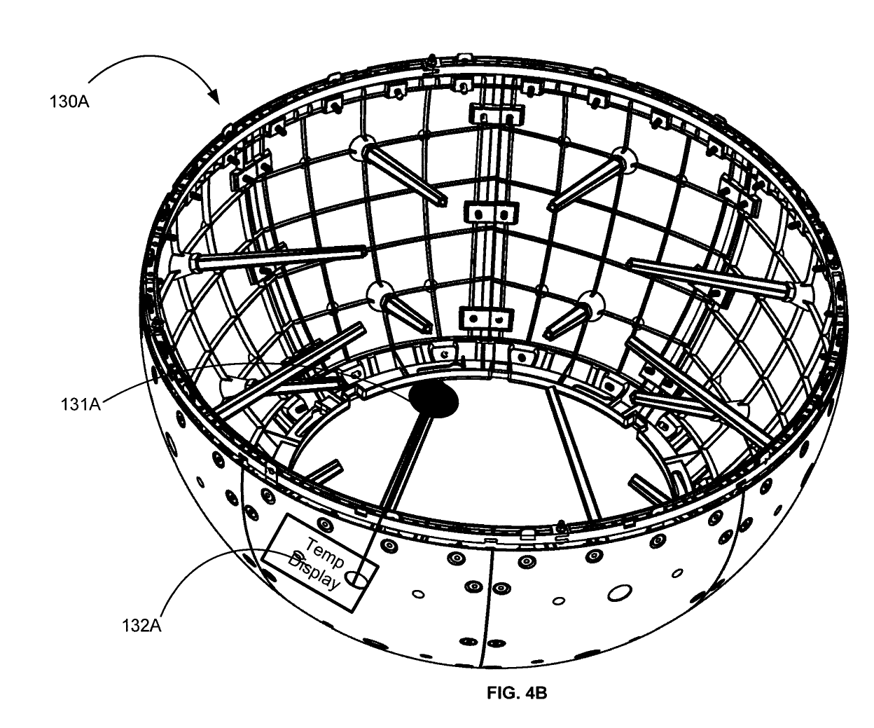

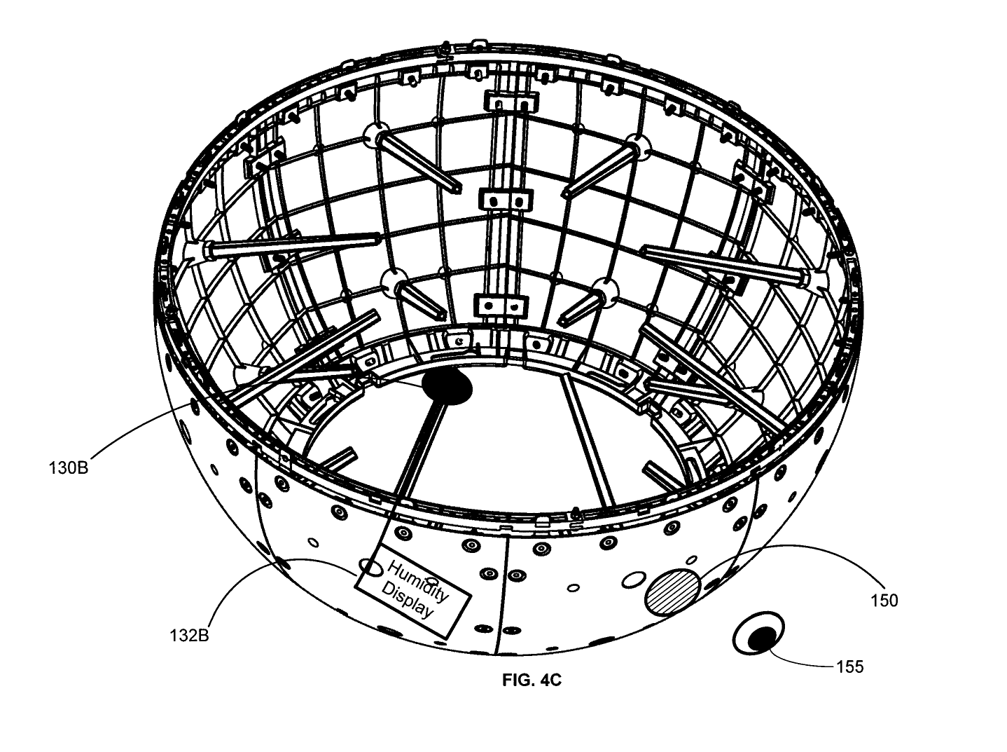

Turning now to FIG. 4A, there is shown an external perspective view with cutaways of a portion of a reactor according to some embodiments of the present invention. According to this embodiment, an exemplary shredding mechanism 200 or assembly 200 includes blades 210, one or more shafts connected through mechanical gears 200, a sleeve 222, a socket 224 and handle 226 for connecting to the socket and turning one of the shafts.

Turning now to FIG. 4B, there is shown an internal perspective view of a portion of a reactor according to some embodiments of the present invention including a temperature reading/sensing apparatus 130A having an internal sensor portion 131A and an external display portion 132A. The sensor portion 131A and the display portion 132A may employ any functionally suitable technology known today or to be devised in the future.

Turning now to FIG. 4C, there is shown an internal perspective view of a portion of a reactor according to some embodiments of the present invention including a humidity reading/sensing apparatus 130B having an internal sensor portion 131B and an external display portion 132B. The sensor portion 131B and the display portion 132B may employ any functionally suitable technology known today or to be devised in the future. Also shown in FIG. 4C is a drainage opening 150 and drainage opening plug 155.

It should be understood by one of skill in the art that some of the functions described as being performed by a specific component of the apparatus may be performed by a different component of the apparatus in other embodiments of this invention.

The present invention can be practiced by employing conventional tools, methodology and components. Accordingly, the details of any such tool, component and methodology are not set forth herein in detail. In the previous descriptions, numerous specific details are set forth, in order to provide a thorough understanding of the present invention. However, it should be recognized that the present invention may be practiced without resorting to the details specifically set forth.

In the description and claims of embodiments of the present invention, each of the words, “comprise” “include” and “have”, and forms thereof, are not necessarily limited to members in a list with which the words may be associated.

Only exemplary embodiments of the present invention and but a few examples of its versatility are shown and described in the present disclosure. It is to be understood that the present invention is capable of use in various other combinations and configurations and is capable of changes or modifications within the scope of the inventive concept as expressed herein.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. An apparatus for composting organic matter, said apparatus comprising:
a reactor adapted to receive organic matter in a reaction chamber of the reactor through a first sealable opening in the reactor, and
at least one protruding structure protruding inward from an inner surface of said reaction chamber, wherein said at least one protruding structure includes a cavity allowing free flow of gases into and out of said reaction chamber.

2. The apparatus according to claim 1, wherein said reactor is of a shape selected from the group of shapes consisting of substantially spherical, substantially oval, substantially egg shaped.

3. The apparatus according to claim 2, wherein said reactor is adapted to rotate along multiple axes.

4. The apparatus according to claim 2, further comprising at least one draining opening in the surface of said reactor, and wherein said at least one draining opening is adapted to allow drainage of fluids from the reaction chamber.

5. The apparatus according to claim 2, wherein said reactor includes a second sealable opening of different diameter from the first sealable opening.

6. The apparatus according to claim 1, further comprising a base structure for supporting said reactor, and wherein said base structure is adapted to allow rotation of said reactor along multiple axes.

7. The apparatus according to claim 6, wherein said base structure further comprises a wheel or ball bearing assembly adapted to facilitate rotation of said reactor along multiple axes.

8. The apparatus according to claim 1, further comprising a shredding mechanism adapted to at least partially shred organic matter introduced into said reaction chamber.

9. The apparatus according to claim 8, wherein said shredding mechanism comprises:
a sleeve passing through a surface of the chamber;
at least one blade, and
a rotatable handle positioned outside said sleeve adapted to rotate said at least one blade.

10. The apparatus according to claim 1 further comprising a sensor and a display.

11. A method for composting organic matter, said method comprising:
introducing organic matter through a sealable opening of a reactor into a reaction chamber wherein composting reaction occurs, and
allowing free flow of gases into and out of said reaction chamber through a cavity of at least one protruding structure protruding inward from an inner surface of said reaction chamber.

12. The method according to claim 11, wherein the reactor is spherical in shape, oval in shape or egg shaped.

13. The method according to claim 12, further comprising rotating the reactor along multiple axes.

14. The method according to claim 12, further comprising drainage of fluids from the reaction chamber through at least one draining opening of the reaction chamber.

15. The method according to claim 12, wherein organic matter is introduced into the reaction chamber through a second sealable opening of different diameter from the first opening.

16. The method according to claim 11, wherein the reactor is supported by a base structure, and the base structure allows rotation of the reactor along multiple axes.

17. The method according to claim 16, wherein the rotation of the reactor along multiple axes is facilitated by at least one wheel in the base structure.

18. The method according to claim 11, further comprising at least partially shredding organic matter introduced into the reaction chamber with a shredding mechanism.

19. The method according to claim 18, wherein shredding comprises rotating at least one blade using a rotatable handle positioned outside the reaction chamber.

20. The method according to claim 11 further comprising sensing and displaying a reaction chamber temperature or a reaction chamber humidity.
